(19) United States
(12) Patent Application Publication
(10) Pub. No.: US 2012/0283335 A1
(43) Pub. Date: Nov. 8, 2012

(54) Analogs of Propofol, Preparation Thereof and Use as Anesthetics

(76) Inventor: Thomas E. Jenkins, San Carlos, CA (US)

(21) Appl. No.: 12/990,486

(22) PCT Filed: May 12, 2009
(86) PCT No.: PCT/US09/43624

§ 371 Date: Jan. 10, 2011

Related U.S. Application Data
(60) Provisional application No. 61/052,474, filed on May 12, 2008.
(60) Provisional application No. 61/052,469, filed on May 12, 2008.
(60) Provisional application No. 61/052,495, filed on May 12, 2008.
(60) Provisional application No. 61/052,504, filed on May 12, 2008.

(51) Int. Cl. A61K 31/05; A61P 25/20; A61P 23/00; C07C 39/06; C07C 37/00

(52) U.S. Cl. 514/731; 568/716; 568/799

(57) ABSTRACT

Compounds of formula (I) wherein X is H or F and pharmaceutically acceptable salts thereof are useful as anesthetics.

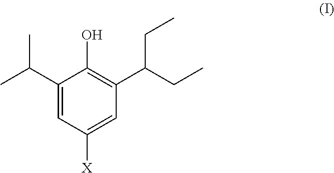

The present application claims the benefit of U.S. provisional patent application No. 61/052,474 filed on May 12, 2008; U.S. provisional patent application No. 61/052,469 filed on May 12, 2008; U.S. provisional patent application No. 61/052,495 filed on May 12, 2008, and U.S. provisional patent application No. 61/052,504 filed on May 12, 2008.

### BACKGROUND OF THE INVENTION

Propofol (2,6-diisopropylphenol) is an intravenous sedative/hypnotic agent used extensively for induction and maintenance of general anesthesia, sedation of critically ill patients and procedural sedation (e.g., endoscopy). See Langly, M. S. and Heel, R. C. Drugs, 1988, 35, 334-372. Propofol is only sparingly soluble in water and is currently marketed in a 10% soybean oil based lipid emulsion similar to formulations used for parenteral nutrition.

Propofol is a GABA_{A} agonist that activates multiple GABA_{A} receptor subtypes, which are ion channels that transport chlorine anions across cell membranes, in the central nervous system. Although propofol is achiral, racemic mixtures of a number of dialkyl phenols are known agonists of the GABA_{A} receptor (James et al., J. Med. Chem. 23, 1350, 1980; Krasowski et al., J. Pharmacol. & Exp. Therapeutics 297, 338, 2001). James et al., report finding propofol to be superior in its overall profile to other analogues evaluated.

Propofol is preferred by many clinicians due to its excellent pharmacokinetic, pharmacodynamic, emergence and recovery profiles. However, undesired side-effects (e.g., respiratory depression, airway collapse, ICU syndrome, injection pain and hemodynamic effects) produced at or near the therapeutic dose greatly limit its utility in multiple clinical settings. Administration of propofol, particularly in bolus form, often produces decreases in blood pressure without a compensatory increase in heart rate. A variety of clinical conditions are incompatible with the use of propofol because of undesired and potentially harmful hemodynamic consequences. Examples of such conditions include cardiovascular disease such as coronary artery disease, cardiomyopathy, ischemic heart disease, valvular heart disease, and congenital heart disease. Chronic hypertension, cerebrovascular disease, brain injury, and advanced age can make the use of propofol difficult or problematic because of its hemodynamic properties. Patients with acute blood loss, dehydration, or severe infection including those with hemorrhagic shock, hypovolemic shock, or septic shock may be exposed to excessive hazard were propofol employed. The hemodynamic properties of propofol may limit its use in patients receiving other medications or treatments such as spinal anesthesia, epidural anesthesia, or vasoactive medications.

Therefore, despite the wide spread success of propofol, there is a need for compounds having an improved profile of properties.

### SUMMARY OF THE INVENTION

The present invention provides certain novel 2,6-dialkylphenols as described in detail hereinafter, which compounds are useful as anesthetics.

### BRIEF DESCRIPTION OF THE DRAWINGS

FIG. 1 shows hemodynamic data for 2-(1-ethylpropyl)-6-isopropylphenol.

FIG. 2 shows hemodynamic data for propofol.

### DETAILED DESCRIPTION OF THE INVENTION

In one aspect, the present invention provides a compound of formula (I):

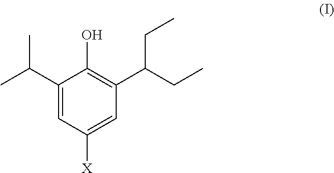

wherein X is H or F; or a pharmaceutically acceptable salt thereof.

In one embodiment, X is H.

The compound in which X is H has the chemical name 2-(1-ethylpropyl)-6-isopropylphenol. Compared with propofol, this compound has been found to exhibit improved safety, improved hemodynamic properties and at least equivalent potency.

In another aspect, the present invention provides a compound of formula (I′):

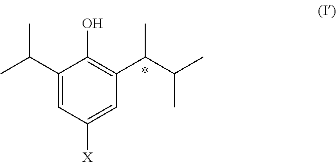

wherein X is H or F, or a pharmaceutically acceptable salt thereof.

A compound of formula (I′) contains a chiral carbon atom, identified by the symbol * in the formula below. It therefore exists and is capable of being isolated in two enantiomeric forms, identified herein as the (−) and (+) stereoisomers.

The present invention provides compounds of formula (I′) in racemic form, and the separate (−) and (+) stereoisomers.

In one embodiment, X is H.

The compound in which X is H has the chemical names 2-(2,3-dimethylpropyl)-6-isopropylphenol and 2-(3-methyl)secbutyl-6-isopropylphenol. Compared with propofol, this compound in racemic form has been found to exhibit improved safety and at least equivalent potency.

In another aspect, the invention provides a (−)-stereoisomer of formula (I″):

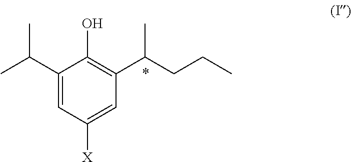

wherein X is H or F; or a pharmaceutically acceptable salt thereof.

In one embodiment, X is H.

The racemic compound in which X is H has the chemical names 2-(3-methylbutyl)-6-isopropylphenol and 2-secpentyl-6-isopropylphenol.

In another aspect, the invention provides a (+)-stereoisomer of formula (I″) wherein X is H or F; or a pharmaceutically acceptable salt thereof.

In one embodiment, X is H.

Stereoisomeric purity of compounds described herein may be established by conventional analytical methods well known to those of skill in the art. For example, use of chiral NMR shift reagents, gas chromatographic analysis using chiral columns, high pressure liquid chromatographic analysis using chiral columns, polarimetry, isotopic dilution, calorimetry, enzymatic methods, capillary electrophoresis on chiral gels, formation of diastereomeric derivatives through reaction with chiral reagents and conventional analysis via established analytical methods may be used to establish the stereochemical purity of a specific stereoisomer. Alternatively, synthesis using starting materials of known stereochemical enrichment may be used to establish the stereochemical purity of the compounds described herein. Other analytical methods for demonstrating stereochemical homogeneity are known in the field.

The present invention provides a stereoisomer of formula (I′) or (I″) or a pharmaceutically acceptable salt thereof in a non-racemic (i.e., an enantiomerically enriched) form at the center marked by “*” in formula (I′) or (I″). Thus the invention includes a stereoisomer of formula (I′) or (I″) in an enriched mixture that contains no more than 45% of the other enantiomer of that compound of formula (I′) or (I″) that is shown or its salt. The (−)-enantiomers and the (+)-enantiomers, the isolation of which is described below, are specific compounds of the invention. In some embodiments of the invention, an enriched mixture contains no more than about 40%, 35%, 30%, 25%, 20%, 15%, 10%, 5%, 4%, 3%, 2%, or 1% of the other enantiomer of a compound of formula (I′) or (I″) or its salt. In another embodiment of the invention an enriched mixture contains less than about 1% of the other enantiomer of a compound of formula (I′) or (I″) or its salt.

Examples of pharmaceutically acceptable salts include salts that are obtained using standard procedures well known in the art, for example by reacting a compound of formula (I), (I′) or (I″) with a suitable base affording a physiologically acceptable cation. For example, alkali metal (for example, sodium, potassium or lithium) or alkaline earth metal (for example calcium) salts can be made.

According to another aspect, the present invention provides a process for preparing a compound of formula (I) or a pharmaceutically acceptable salt thereof, which comprises reducing a compound of general formula (II)

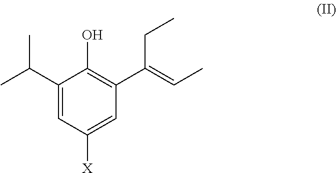

in which X is H or F, or a salt thereof, followed if desired by forming a pharmaceutically acceptable salt.

The reduction is conveniently effected by hydrogenation in the presence of a group VIII metal catalyst, such as palladium on carbon.

Compounds of formula (II) may be prepared by dehydrating a compound of general formula (III)

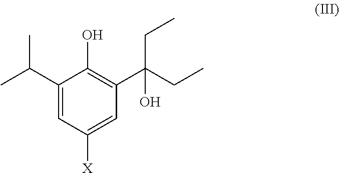

in which X is H or F, or a salt thereof.

The dehydration is conveniently effected in the presence of a dehydrating agent, for example an acid such as hydrochloric acid or p-toluenesulfonic acid.

Compounds of general formula (III) may be prepared by reacting a compound of general formula (IV) or (V)

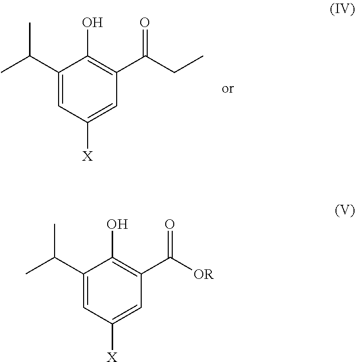

in which OR represents a residue of an alcohol, for example a (1-6C)alkanol, with a Grignard reagent, such as ethyl magnesium bromide.

According to another aspect, the present invention provides a process for preparing a compound of formula (I′) or a (−) or (+) stereoisomer or a pharmaceutically acceptable salt thereof, which comprises reducing a compound of general formula (II′)

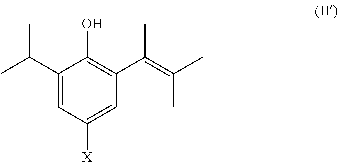

in which X is H or F, or a salt thereof, followed if desired by isolating a (−) or (+) stereoisomer and/or forming a pharmaceutically acceptable salt.

The reduction is conveniently effected by hydrogenation in the presence of a group VIII metal catalyst, such as palladium on carbon.

Compounds of formula (II′) may be prepared by dehydrating a compound of general formula (III′)

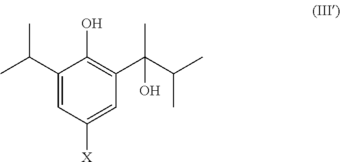

in which X is H or F, or a salt thereof.

The dehydration is conveniently effected in the presence of a dehydrating agent, for example an acid such as hydrochloric acid or p-toluenesulfonic acid.

Compounds of general formula (III′) may be prepared by reacting a compound of general formula (IV′)

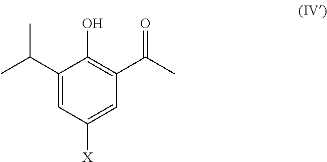

with a Grignard reagent, such as isopropyl magnesium bromide.

Compounds of general formula (IV′) may be prepared by reacting a compound of formula (V′)

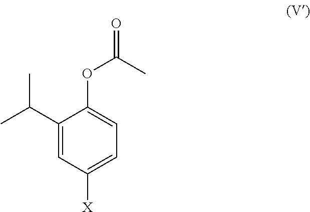

with a Lewis acid, such as aluminum trichloride.

Compounds of formula (V′) may be prepared from the corresponding 2-isopropylphenol by reaction with acetic anhydride.

The (−) and (+) stereoisomers may be isolated using methods known in the art for separating enantiomers. Examples of such methods include separation on a chiral column, either as the phenol itself or as a derivative, such as an ester, carbonate or carbamate, from which the desired stereoisomer can be liberated, for example by hydrolysis.

According to another aspect, the present invention provides a process for preparing a (−)-stereoisomer of formula (I″) or a pharmaceutically acceptable salt thereof, which comprises reducing a compound of general formula (II″)

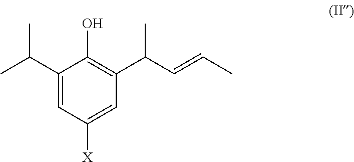

in which X is H or F, or a salt thereof, followed if necessary by isolating the desired stereoisomer and/or forming a pharmaceutically acceptable salt.

The reduction is conveniently effected by hydrogenation in the presence of a group VIII metal catalyst, such as palladium on carbon.

Compounds of formula (II″) may be prepared by rearranging a compound of general formula (III″)

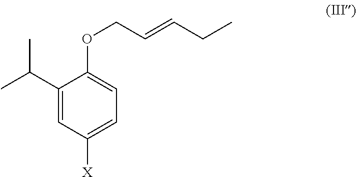

in which X is H or F, or a salt thereof.

The rearrangement is conveniently effected by heating a compound of formula (III″) to a temperature of greater than 200° C.

Compounds of formula (III″) can be prepared by reacting 2-isopropylphenol with 2-penten-1-ol in the presence of a triphenylphosphosphine and diisopropyl azodicarboxylate (DIAD).

The (−)-stereoisomers and the (+) stereoisomers may be isolated using methods known in the art for separating enantiomers. Examples of such methods include separation on a chiral column, either as the phenol itself or as a derivative, such as an ester or carbamate, from which the desired stereoisomer can be liberated, for example by hydrolysis. An example of an ester is an aryl ester, such as the benzoyl ester. An example of a carbamate is a carbamate derived from a chiral amine, such as a chiral 1-phenylethylamine. Chiral HPLC columns may be obtained, for example, from Daicel, Inc in the USA. An example of a chiral HPLC column is Daicel, Inc. CHIRALCEL ODH 20×250 millimeter (mm), 5 micron (μm). Suitable solvents for the separation include HPLC grade n-hexane as the mobile phase.

The compounds according to the invention (i.e. a compound of formula (I), (I′) or (I″) or a pharmaceutically acceptable salt thereof) are generally formulated in a pharmaceutical composition for administration to a patient.

According to another aspect, therefore, the present invention provides a pharmaceutical composition, which comprises a compound of the invention and a pharmaceutically acceptable carrier.

As used herein, the term “pharmaceutically acceptable carrier” includes diluents, adjuvants, excipients or vehicles.

The compounds of the invention may be formulated as pharmaceutical compositions and administered to a patient, in a variety of forms adapted to the chosen route of administration, e.g., orally, parenterally, intravenously, intramuscularly, topically, subcutaneously or by inhalation.

Thus, the compounds of the invention can be systemically administered, in combination with pharmaceutically acceptable carriers such as inert diluents or edible carriers. Such compositions and preparations may contain at least 0.1% of active compound. The percentage of the compositions and preparations can, of course, be varied and can conveniently be between about 0.1% to about 60% of the weight of a given unit dosage form. The amount of active compound in such therapeutically useful compositions is such that an effective dosage level is obtained.

The compounds of the invention described herein are typically formulated as pharmaceutical compositions suitable for intravenous administration. The compounds of the invention may be relatively insoluble in water. Thus, for intravenous administration, the compounds of the invention are typically formulated in aqueous media using one or more water-immiscible solvents and one or more emulsifiers or surfactants. Individual formulations can include one or more additional components such as stabilizers, tonicity modifiers, bases or acids to adjust pH and solubilizers. The formulations may also optionally contain a preservative, such as, for example, ethylenediaminetetraacetic acid (EDTA) or sodium metabisulfite. Useful oil-in-water emulsions that contain a preservative such as EDTA that may be used in conjunction with compounds described herein are described in U.S. Pat. Nos. 5,908,869, 5,714,520, 5,731,356 and 5,731,355.

A wide range of water-immiscible solvents can be used in the pharmaceutical compositions described herein. The water-immiscible solvent can be a vegetable oil, such as, for example, soybean, safflower, cottonseed, corn, sunflower, arachis, castor or olive oil. Alternatively, the water-immiscible solvent may be an ester of a medium or long-chain fatty acid, such as, for example, a mono-, di-, or triglyceride, an ester of a combination of a medium and long-chain fatty acid or a chemically modified or manufactured material such as ethyl oleate, isopropyl myristate, isopropyl palmirate, a glycerol ester, polyoxyl or hydrogenated castor oil. The water-immiscible solvent can also be a marine oil, such as, for example, cod liver or another fish-derived oil. Other suitable solvents include fractionated oils, such as, for example, fractionated coconut oil or modified soybean oil. The water-immiscible solvent may include “structured lipids.” (see, e.g., Lipid Biotechnology, T. M. Kuo and H. W. Gardner (eds.), Marcel Dekker, Inc. New York, N.Y.). Many structured lipids are available from commercial suppliers such as Danisco AIS, Copenhagen Denmark and S&J Lipids, Ostrander, Ohio.

The pharmaceutical compositions described herein can also contain an emulsifier. Suitable emulsifiers include synthetic non-ionic emulsifiers, such as, for example, ethoxylated ethers, ethoxylated esters, polyoxypropylene-polyoxyethylene block co-polymers and phospholipids. Naturally-occurring phospholipids, such as egg or soya phospholipids, and modified or artificially manipulated phospholipids or mixtures thereof can also be used. In some embodiments, emulsifiers are egg phospholipids and soya phospholipids. Egg yolk phospholipids include phosphatidylcholine, lecithin and phosphatidylethanolamine.

The pharmaceutical formulations described herein can comprise a lipid emulsion comprising from about 0.1% to about 5% (w/w) of a compound of the invention, from about 5 to about 25% (w/w) water immiscible solvent and from about 40% to about 90% (w/w) water. A preferred formulation comprises from about 0.5% to about 2% (w/w) of a compound of the invention. In one embodiment, a pharmaceutical formulation comprises from about 0.5% to about 5% (w/w) of a compound of the invention and from about 0% to about 50% (w/w) of a water immiscible solvent.

The pharmaceutical formulations described herein may also include stabilizing agents. Anionic stabilizers include, for example, phosphatidylethanolamines, conjugated with polyethylene glycol, (PEG-PE) and phosphatidylglycerols, a specific example of which is dimyristolphosphatidylgylcerol (DMPG). Additional stabilizers include, but are not limited to, oleic acid and its sodium salt, cholic acid and deoxycholic acid and respective salts thereof, cationic lipids such as stearylamine and oleylamine, and 3|3-[N—(N′,N′-dimethylaminoethane)carbamoyl]cholesterol (DC-Chol).

The pharmaceutical compositions described herein can be made isotonic with blood by the incorporation of a suitable tonicity modifier. Glycerol is most frequently used as a tonicity modifier. Alternative tonicity modifying agents include xylitol, mannitol and sorbitol. The pharmaceutical compositions are typically formulated to be at physiologically neutral pH, typically in the range 6.0-8.5. The pH can be adjusted by the addition of base, for example, NaOH or NaHCO₃, or in some cases acid, such as HCl.

The compounds of the invention can be formulated with pharmaceutically safe oil-water emulsions comprising a vegetable oil, a phosphatide emulsifier, typically egg lecithin or soybean lecithin, and a tonicity modifier such as, for example, Liposyn® II and Liposyn® III (Abbott Laboratories, North Chicago, Ill.) and Intralipid® (Fresenius Kabi AB, Uppsala, Sweden) or other similar oil-water emulsions.

Compounds of the invention can also be formulated in a triglyceride comprising esters of at least one medium chain length (C₆-C₁₂) fatty acid. In some embodiments, the triglyceride is an ester of a C₈-C₁₀ fatty acid. Triglycerides suitable for formulating compounds of the invention include, but are not limited to, Miglyol® (Condea Chemie GmbH, Witten, Germany). For example, Miglyol® 810 or 812 (caprylic (C₁₀)/capric (C₈) glyceride) is useful for formulation of compounds of the invention.

Additionally, compounds of the invention described herein can be formulated analogously to pharmaceutical compositions of propofol as described, for example, in U.S. Pat. Nos. 4,056,635, 4,452,817 and 4,798,846.

Still other suitable formulations for use in the present invention can be found, for example in Remington's Pharmaceutical Sciences, Philadelphia, Pa., 19th ed. (1995).

A compound of the invention and/or pharmaceutical compositions thereof may be administered alone or in combination with other pharmaceutical agents including compounds disclosed herein and/or pharmaceutical compositions thereof. The compounds disclosed herein may be administered or applied per se or as pharmaceutical compositions. The specific pharmaceutical composition depends on the desired mode of administration, as is well known to the skilled artisan.

Compounds disclosed herein and/or pharmaceutical compositions thereof may be administered to a subject by intravenous bolus injection, continuous intravenous infusion, oral tablet, oral capsule, oral solution, intramuscular injection, subcutaneous injection, transdermal absorption, buccal absorption, intranasal absorption, inhalation, sublingually, intracerebrally, intravaginally, rectally, topically, particularly to the ears, nose, eyes, or skin or any other convenient method known to those of skill in the art. In some embodiments, compounds disclosed herein and/or pharmaceutical compositions thereof are delivered via sustained release dosage forms, including oral sustained release dosage forms. Administration can be systemic or local. Various delivery systems are known, (e.g., encapsulation in liposomes, microparticles, microcapsules, capsules, “patient controlled anesthesia” drug delivery systems, etc.) that can be used to deliver compounds disclosed herein and/or pharmaceutical compositions thereof.

In certain embodiments, compounds disclosed herein and/or pharmaceutical compositions thereof can be used in combination therapy with at least one other therapeutic agent. The compounds disclosed herein and/or pharmaceutical compositions thereof and the therapeutic agent can act additively or, more preferably, synergistically. In some embodiments, compounds disclosed herein and/or pharmaceutical compositions thereof are administered concurrently with the administration of another therapeutic agent such as, for example, other sedative hypnotic agents (e.g., etomidate, thiopental, midazolam, dexmedetomidine, ketamine), anesthetic agents (e.g., desflurane, sevoflurane, isoflurane, nitrous oxide), analgesics (e.g., an opioid such as remifentanil, morphine, meperidine, hydromorphone, methadone, fentanyl, sulfentanil, or alfentanil, or a non-opioid analgesic such as ketorolac, gapapentin, lidocaine, or ketamine), paralytic agents, such as rocuronium, cis-atracurium, vecuronium, or pancuronium bromide, anti-emetics (e.g., ondansetron, dolasetron, droperidol), cardiovascular agents (e.g., metoprolol, propranolol, esmolol, clonidine, phenylephrine, ephedrine, epinephrine, norepineprine, dopamine, diltiazem, atropine, glycopyrrolate, lisinopril, nitroglycerin, sodium nitroprusside, digoxin, milrinone), steroids (e.g., dexamethasone, hydrocortisone, methylprednisolone), anti-infective agents (e.g., cefazolin, vancomycin), diuretics (e.g., furosemide, hydrochlorothiazide, spironolactone), mood altering medications (e.g., fluoxetine, aripiprazole), or stimulants such as nicotine or cytisine.

For example, compounds disclosed herein and/or pharmaceutical compositions thereof may be administered together with other therapeutic agents. In other embodiments, compounds disclosed herein and/or pharmaceutical compositions thereof are administered prior or subsequent to administration of other therapeutic agents.

The compounds of the invention are useful as anesthetics and sedatives.

According to another aspect, the present invention provides a method for inducing or maintaining general anesthesia in an animal comprising administering to the animal an effective amount of a compound of the invention.

According to yet another aspect, the present invention provides a method for promoting sedation in an animal comprising administering to the animal an effective amount of a compound of the invention.

The animal, or patient, may be a human or non-human animal, such as a companion animal or a zoo animal, for example a dog, cat or horse.

The term “effective amount” indicates the amount effective to produce the desired effect; i.e. to induce or maintain anesthesia, or promote sedation.

The amount of compounds disclosed herein and/or pharmaceutical compositions thereof that will be effective can be determined by standard clinical techniques known in the art. The amount of compounds disclosed herein and/or pharmaceutical compositions thereof administered will, of course, be dependent on, among other factors, the subject being treated, the weight of the subject, the age of the subject, the condition of the subject, the intended effect of the compounds, the manner of administration and the judgment of the prescribing physician. For example, the dosage level of a compound of the invention for producing general anesthesia may be in the range of from about 1 to about 12 mg/kg. Preferred induction doses range from about 1 to about 4 mg/kg. Preferred maintenance doses range from about 1 to about 30 mg/kg/hr. Preferred doses to produce a sedative effect range from about 0.3 to about 12 mg/kg/hr.

The compounds of the invention are believed to act like propofol as agonists at GABA receptors. Accordingly, they are useful in the treatment of a variety of diseases or disorders, including nausea, vomiting, migraine, anxiety, insomnia, neurodegenerative conditions of the nervous system (e.g., Friedrich's disease, Parkinson's disease, Alzheimer's disease, Huntington's disease, amyotrophic lateral sclerosis (ALS), multiple sclerosis (MS), Pick disease, etc.), trauma to the central nervous system (e.g., skull fracture and its resulting edema, concussion, contusion, brain hemorrhages, shearing lesions, subdural and epidural hematoma, and spinal cord injury (e.g., mechanical injury due to compression or flexion of the spinal cord)), seizures (e.g., epileptic seizures) or a free radical associated disease (e.g., ischemic reperfusion injury, inflammatory diseases, systemic lupus erythematosis, myocardial infarction, stroke, traumatic hemorrhage, cataract formation, uveitis, emphysema, gastric ulcers, neoplasia, radiation sickness, etc.). The present invention also provides a method of treating each of these conditions in an animal comprising administering an effective amount of a compound of the invention to the animal.

“Treating” a disease or disorder includes 1) ameliorating the disease or disorder (i.e., arresting or reducing the development of the disease or disorder or at least one of the clinical symptoms thereof), 2) ameliorating at least one physical parameter, which may not be discernible by the patient, 3) inhibiting the disease or disorder which can be either physically, (e.g., stabilization of a discernible symptom), physiologically, (e.g., stabilization of a physical parameter) or both, or 4) delaying the onset of the disease or disorder.

The invention also provides a compound of the invention for use in therapy.

The invention further provides a compound of the invention for inducing or maintaining general anesthesia in an animal.

The invention also provides a compound of the invention for promoting sedation in an animal.

The ability of a compound of the invention to produce an anesthetic effect (i.e. efficacy) can be determined using standard pharmacological models that are well known to the art. The potency of the anesthetic effect of a compound of the invention was demonstrated in a loss of righting reflex assay in the rat (as described in Test A below). The potency of such a compound was compared to the potency of propofol using this assay.

The safety of a compound of the invention can be determined using standard pharmacological models that are well known to the art. The safety of a compound of the invention was demonstrated by evaluating the maximum tolerated dose (MTD) in the rat (as described in Test A below). The safety of such a compound was compared to that of propofol using this assay.

The hemodynamic profile of a compound of the invention can be determined using standard pharmacological models that are well known to the art. The hemodynamic and anesthetic profiles of a compound of the invention can be evaluated simultaneously using an anesthetized pig model (as described in Test B below).

### Test A. Loss of Righting Reflex Assay

Male Sprague Dawley rats were restrained in a holder, and test compound or propofol was injected in the tail vein (based on milligrams (mg) compound per kilogram (kg) body weight). Following administration, the rats were placed in a dorsal recumbent position on a heating blanket. The time to onset for the loss of the Righting Reflex (RR-ability of rat to right itself) was recorded, as was the duration of the loss of the RR. Doses were escalated until the maximum tolerated doses were achieved.

2-(1-Ethylpropyl)-6-isopropylphenol was shown to have at least equivalent, if not greater, potency than propofol at an equal dose (7 mg/kg) with similar rapid onset. Yet, when doses were escalated, 2-(1-ethylpropyl)-6-isopropylphenol was shown to have greater safety compared to propofol. Representative data are shown in Table 1.

TABLE 1
Safety of 2-(1-ethylpropyl)-6-isopropylphenol
compared to propofol

| | Outcome | |
|---|---|---|
| | | 2-(1-ethylpropyl)-6- |
| Bolus Dose (mg/kg) | Propofol | isopropylphenol |
| 7 | 0/10 deaths | 0/6 deaths |
| | 7.1 minutes of sleep post | 9.9 minutes of sleep post |
| | bolus dose | bolus dose |
| 14 | 6/10 deaths | 0/6 deaths |
| 21 | 4/6 deaths | 0/6 deaths |

2-(3-methyl)secbutyl-6-isopropylphenol was shown to have at least equivalent, if not greater potency than propofol at an equal dose (7 mg/kg) with similar rapid onset. Yet, when doses were escalated, 2-(3-methyl)secbutyl-6-isopropylphenol, was shown to have greater safety compared to propofol. Representative data are shown in Table 2.

TABLE 2
Safety of racemic 2-(3-methyl)secbutyl-6-isopropylphenol
compared to propofol

| | Outcome | |
|---|---|---|
| | | 2-(3-methyl)secbuty1-6- |
| Bolus Dose (mg/kg) | Propofol | isopropylphenol |
| 7 | 0/10 deaths | 0/6 deaths |
| | 7.1 minutes of sleep post | 7.5 minutes of sleep post |
| | bolus dose | bolus dose |
| 14 | 6/10 deaths | 0/6 deaths |
| 21 | 4/6 deaths | 0/6 deaths |

### Test B. Anesthetized Pig Model

The anesthetic induction was performed using a modification of a technique described by Ko et al (Ko et al., Lab Anim Sci 1993; 43: 476-80) for swine (Telazol, xylazine and ketamine given as an intramuscular injection). The minimal effective dose for induction and tracheal intubation was used. When the animal was recumbent, oxygen was administered by mask at 8 mL/min and an IV was started in an ear vein running normal saline at 70 mL/hr. The pig's trachea was intubated and mechanically ventilated to maintain arterial P_{CO2} at approximately 35 mmHg.

Electrocardiogram (ECG) electrodes were placed using a lead II configuration to monitor cardiac activity. An arterial catheter was placed in the right femoral artery to monitor blood pressure. A pulmonary artery catheter was placed via the right jugular vein for measuring cardiac output, pulmonary capillary wedge pressure, and central venous pressure. A catheter was also placed in the abdominal aorta via the left femoral artery for blood sampling.

Bipolar electroencephalograph (EEG) leads were placed using low impedance surface electrodes placed over the frontal and occipital regions of the cerebral hemispheres, approximately 50 mm apart and 20 mm from the midline. A ground electrode was placed midline between the frontal and occipital regions. Alternatively, an integrated electrode sensor array (Aspect Medical) compatible with an electroencephalogram analyzer (Aspect Medical) was applied. Anesthesia was maintained with isoflurane adjusted to keep the mean arterial blood pressure at 100 millimeters of mercury (mmHg or mm Hg) during the stabilization period and intravenous pancuronium was administered as needed for muscle relaxation.

After the initial animal instrumentation was complete, (usually requiring approximately 2-3 hours), an additional 1 hour and 15 minutes served as a period of stabilization and baseline data gathering (and to ensure near complete dissipation of the effects of the anesthetic induction drugs). Isoflurane inhalation was continued throughout the study or stopped, and the isoflurane was allowed to “wash-out” for 15 mins prior to the administration of a compound of the invention.

A compound of the invention or propofol was administered intravenously by a 20 minute infusion after the stabilization period through the peripheral IV catheter. A pilot dose-finding study was performed to establish an appropriate infusion dose for each compound. In this pilot study, multiple doses (up to 5 infusions in total) were administered to each pig with at least 90 minutes between doses. Blood samples (1 mL each) were collected at pre-dose and at 2, 4, 6, 8, 10, 12, 15, 20, 22, 22, 25, 35, 50, 65 and 80 minutes after the start of the first infusion for pharmacokinetic purposes; EEG were recorded continuously as the primary pharmacodynamic endpoint.

Arterial blood samples (1 mL each) were taken from the abdominal aorta at 2, 4, 6, 8, 10, 11, 12, 13, 14, 15, 17.5, 20, 25, 30, 45, 60, 90, 120 and 180 minutes after the start of the infusion. A control sample prior to the start of the infusion was also taken.

The EEG signal was fed to a BIS analyzer (Aspect Medical) which provided continuous output of processed EEG data. The output consisted of a “BIS” number calculated by a proprietary algorithm that ranges between 100 (fully conscious) and 0 (isoelectric) and indicated brain activity.

Hemodynamic data were recorded and plotted to assess trends over the drug exposure period. Data for each compound of the invention were compared to propofol for effects on BIS, heart rate, mean arterial blood pressure and cardiac output.

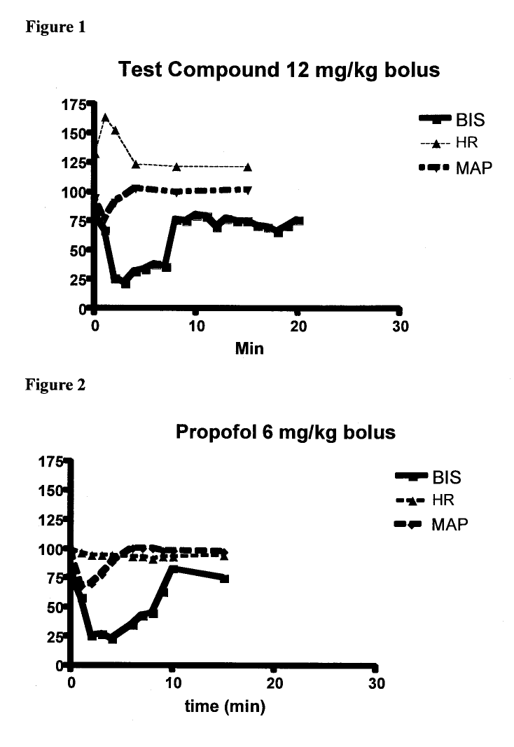

Hemodynamic data for 2-(1-ethylpropyl)-6-isopropylphenol and for propofol are shown in FIGS. 1 and 2.

The invention will now be illustrated by the following non-limiting Examples.

### Example 1

### Synthesis of 2-(1-ethylpropyl)-6-isopropylphenol

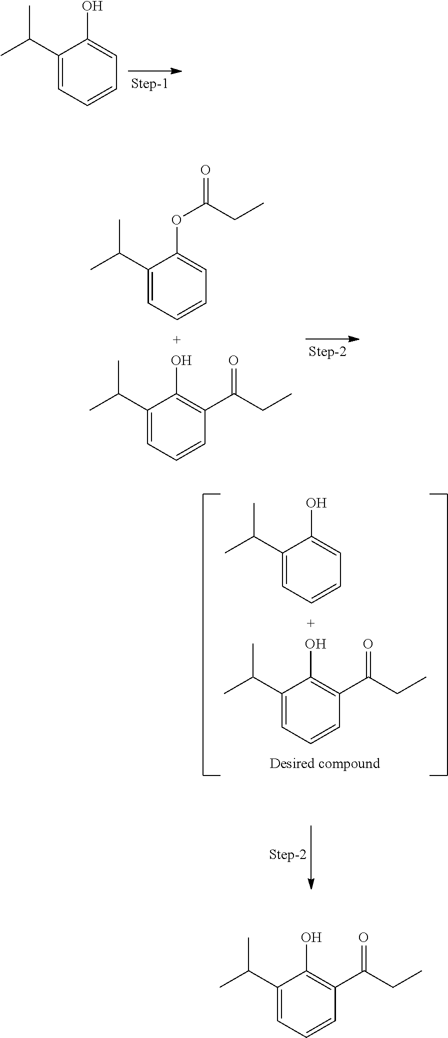

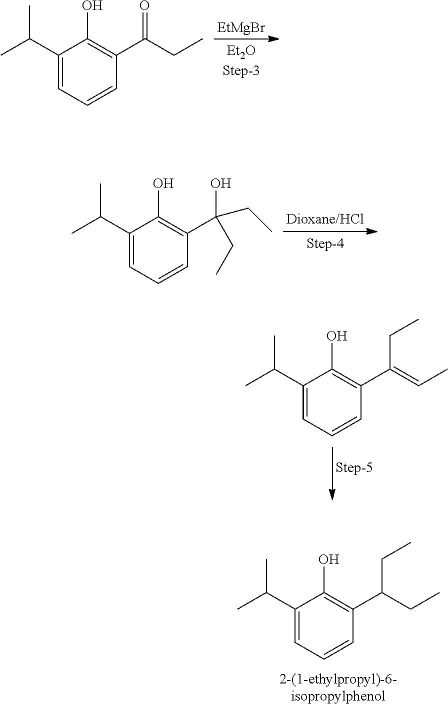

### Step-1:

To a solution of 2-Isopropylphenol (20 grams (g), 0.133 mole (mol)) in dry toluene (150 milliliters (ml or mL)) was added trimethyl aluminum (23 ml, 0.045 mol) at 0° C., followed by a solution of propionyl chloride (16.4 ml, 0.160 mol) under a nitrogen atmosphere. The mixture was then stirred at ambient temperature overnight. Saturated ammonium chloride (NH₄Cl) solution (20 ml) was then added, and the resultant mixture was extracted with ethyl acetate (EtOAc) (2×100 ml). The organic layer was washed with water (100 ml) and brine (1×100 ml), dried over anhydrous sodium sulfate (Na₂SO₄) and concentrated. The crude material was purified by column chromatography using 2% EtOAc in petroleum ether to result in a mixture of products (5 g).

Step-2 2-propanoyl-6-isopropylphenol:

To the mixture of products from step-1 (13 g, 0.063 mol) in tetrahydrofuran (THF) (100 ml) was added a solution of lithium hydroxide (3.02 g, 0.126 mol) in water (10 ml) and the resultant mixture was stirred at ambient temperature overnight. The reaction mixture was then acidified with 1.5 normal (N) hydrochloric acid (HCl) and extracted with EtOAc (150 ml). The organic layer was washed with water (100 ml) and brine (100 ml), dried over anhydrous Na₂SO₄ and concentrated. The crude material was purified by column chromatography using 2% EtOAc in petroleum ether to afford step-2 product (ortho isomer) as colorless liquid (0.4 g, 2%).

Step-3 2-(1-ethyl-1-hydroxypropyl)-6-isopropylphenol:

To a solution of the product of step-2 (0.4 g, 0.001 mol) in dry diethyl ether (10 ml) was added ethyl magnesium bromide (4.21 ml, 0.002 mol) at 0° C. under nitrogen. The resultant mixture was stirred at ambient temperature for 3 hours (h, hr, or hrs). The reaction mixture was then cooled to 0° C. 1.5 N HCl (3 ml) was then added and the resultant mixture was extracted with ethyl acetate (50 ml). The organic layer was washed with water (20 ml) and brine (20 ml), dried over sodium sulfate and concentrated to afford the product as yellow liquid (0.13 g, 29%).

Step-4: 2-(1-ethylprop-1-enyl)-6-isopropylphenol

A solution of the product of step-3 (0.13 g, 0.0005 mol) in dry dioxane (5 ml) was cooled to 0° C., purged with dry HCl gas for 30 minutes (min) and then stirred at ambient temperature for 2 h. The reaction mixture was then concentrated to obtain a product as yellow liquid (0.1 g, 83.3%).

Step-5 2-(1-ethylpropyl)-6-isopropylphenol:

To the product of step-4 (0.1 g, 0.0004 mol) dissolved in dry methanol (5 ml) was added palladium on carbon (Pd/C) (0.015 g, 10 mol %) under nitrogen. The mixture was then hydrogenated at 4 kg pressure. The reaction mixture was then filtered through CELITE (Celite Corporation) and the filtrate concentrated under reduced pressure. The crude material was purified by column chromatography using 2% EtOAc in petroleum ether to afford 2-(1-ethylpropyl)-6-isopropylphenol as colorless liquid (0.08 g, 74%).

### Example 2

### Synthesis of 2-(1-ethylpropyl)-6-isopropylphenol

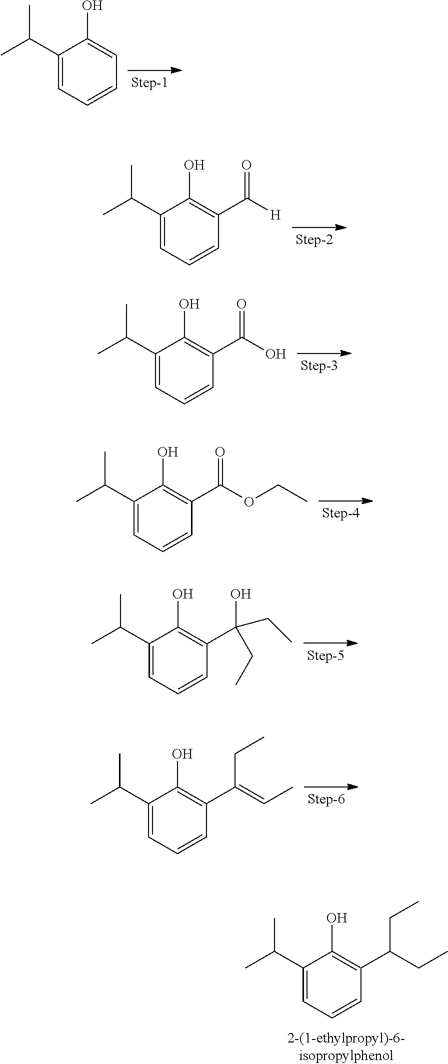

Step-1 2-formyl-6-isopropylphenol.

To a mixture of dry anhydrous magnesium dichloride (139.0 gm, 1.46 mol) and solid paraformaldehyde (63.5 gm, 2.19 mol) under an argon atmosphere were added dry tetrahydrofuran (2.5 lit) followed by triethylamine (203.0 ml, 1.46 mol) drop wise over 20 min at ambient temperature with stirring. The reaction was further stirred for 10 min. 2-Isopropylphenol was then added drop wise over 20 min at ambient temperature. The mixture was then heated slowly to reflux for 4 hrs. The reaction mixture was then cooled to ambient temperature and 1.5 liters (l or lit) of ether was added. The resulting organic phase was transferred to a separatory funnel and washed successively with 1 N HCl (1.5 lit two times (×2)) and water (1.5 l×2), dried over anhydrous sodium sulfate, and filtered. The solvent was removed by rotatory evaporation; affording a pale yellow oil (115.0 gm, 94.3%).

Step-2 2-carboxy-6-isopropylphenol.

To the stirred solution of the product of step 1 (115 g, 0.7 mol) in ethanol (1150 ml), was added silver nitrate (AgNO₃) followed by the 80% Ethanolic-sodium hydroxide (NaOH) solution in drop wise manner over 5 to 6 hrs at ambient temperature. The reaction mixture was further stirred at ambient temperature for 1 hr. The reaction was monitored by thin layer chromatography (TLC). After completion of the reaction, the reaction mixture was filtered and the filtrate acidified with concentrated HCl to pH #2 at 10-15° C. The reaction mixture was then filtered, and the filtrate evaporated. The residue was dissolved in ethyl acetate (500 ml) and water (500 ml), and the aqueous and organic layers separated. The aqueous layer was re-extracted with ethyl acetate (500 ml). The combined organic layers were dried over anhydrous Na₂SO₄, filtered and the solvent evaporated to yield the crude acid (80 g, 63%).

Step-3 2-ethoxycarbonyl-6-isopropylphenol.

To the stirred solution of the product of step 2 (80.0 g, 0.44 mol) in ethanol (2000 ml), was added hydrogen sulfate (H₂SO₄) (16 ml) drop wise at ambient temperature. The reaction mixture was heated under reflux for 48 hr. The reaction was monitored by TLC. The solvent was then evaporated to yield a crude oil. The oil was dissolved in ether and the ether layer was washed with water, dried over anhydrous Na₂SO₄, filtered and the solvent evaporated to yield a brown oil. The ester was purified by silica gel column chromatography using ethyl acetate and hexane (20 g, 21.6%).

Step-4 2-(1-ethyl-1-hydroxypropyl)-6-isopropylphenol.

### Formation of Grignard Reagent.

To the stirred solution, magnesium (Mg) (11.3.0 g, 0.464 mol) in ether (200 ml) was added a small amount of iodine (I₂) under a nitrogen (N₂) atmosphere followed by the addition of ethyl bromide drop wise at ambient temperature. After completion of the addition, the reaction was stirred at ambient temperature for 1 hr.

### Formation of the Alcohol.

To a solution of the product of step 3 in ether under N₂ was added the Grignard reagent drop wise at 0° C. After completion of the addition, the reaction mixture was heated under reflux for 1 hr. The reaction was monitored by TLC. To the reaction was then added a saturated solution of NH₄Cl aqueous (aq.) at 0° C. drop wise. The ether layer was separated and dried over anhydrous Na₂SO₄, filtered and the solvent evaporated to yield an oil (20 g, 93.6%).

Step-5 2-(1-ethylprop-1-enyl)-6-isopropylphenol.

To the stirred solution of the product of step 4 (20.0 g, 0.09 mol) in MDC (200 ml) was added p-toluenesulfonic acid (PTSA) (18.82 g) at ambient temperature. The reaction was stirred at ambient temperature for 2 h, and monitored by TLC. Water (200 ml) was then added and the organic layer separated and dried over anhydrous Na₂SO₄, filtered, and the solvent evaporated to yield an oil (17.7 g, 88%).

Step-6 2-(1-ethylpropyl)-6-isopropylphenol.

To the stirred solution of the product of step 4 (17.7 g, 0.086 mol) in THF (170 ml) in Parr apparatus bottle was added 10% Pd/C (17.7 g) at ambient temperature. Hydrogen (H₂) was applied at a pressure of up to 4 to 5 kg in Parr bottle for 3 to 4 h. The reaction was monitored by TLC. The catalyst was then filtered off and the solvent was evaporated to yield a crude oil from which the product was purified by silica gel column chromatography using hexane and ethyl acetate to afford the title compound (11.0 g, 62%).

NMR (Instrument: Bruker AVANCE 300 MHz. Solvent: deuterated chloroform (CDCl₃) Sample Preparation: 2.0 mg sample was dissolved in 0.75 ml CDCl₃) consistent with assigned structure.

LCMS (Instrument: AB SCIEX-2000 API (Applied Biosystems). Mode: −Ve (Negative), Sample Preparation: 1.0 mg sample was dissolved in 50.0 ml methanol and spectra was recorded using sample infusion technique) also consistent with assigned structure. M−1=205.3; calculated mass=206.2.

### Example 3

### Synthesis of 2-(3-methyl)secbutyl-6-isopropylphenol

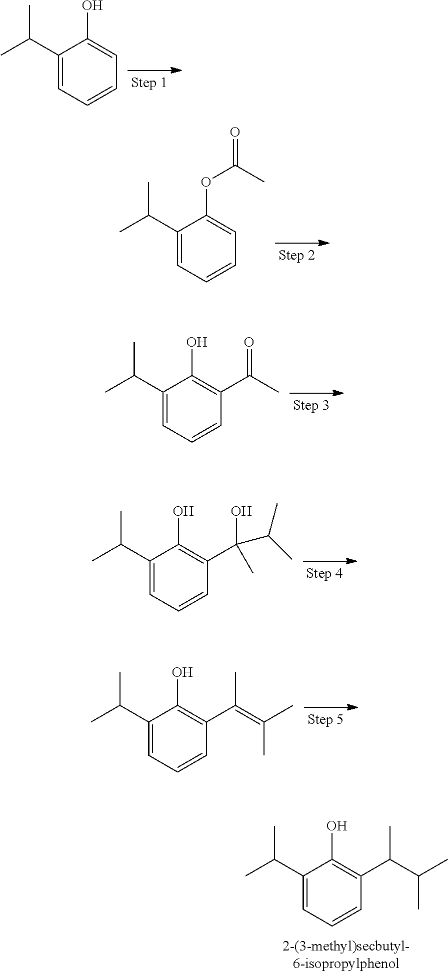

### Step-1

### Synthesis of Compound 2:

To the stirred solution of 2-Isopropylphenol (105 grams (g), 0.77 moles (mol)) in MDC (700 milliliters (ml or mL)), was added charged 4-dimethylaminopyridine (DMAP) (112.9 g, 0.92 mol) followed by acetic anhydride (91 ml, 1.0 mol) at ambient temperature. The reaction was stirred at ambient temperature for 2 hours (h, hr or hrs). The solvent was then distilled off and the residue was dissolved in ether. The ether layer was washed with 2 normal (N) hydrochloric acid (HCl) solution, sodium bicarbonate (NaHCO₃) aqueous (aq.), brine solution, and dried with anhydrous sodium sulfate (Na₂SO₄). The ether was evaporated to yield a pale yellow oil (135 g, 98.3%).

### Step-2 Synthesis of Compound 3:

To the stirred solution of aluminum chloride (AlCl₃) in carbon disulphide (450 ml), was added a solution of compound 2 (135 g, 0.75 mol) in CS₂ dropwise at ambient temperature. The reaction was stirred at ambient temperature for 1 hr, and then heated under reflux for 2 hrs. The solvent was evaporated and the reaction then heated at 70° C. for 6 hrs. The reaction was monitored by thin layer chromatography (TLC). The reaction was then cooled to ambient temperature and added to dilute HCl aq. The compound was extracted by the addition of ether, and the ether layer washed the with NaHCO₃ aq. followed by brine. The ether was evaporated to yield a crude product of 126 g. The crude product was purified by silica gel column chromatography by using Hexane & Ethyl acetate to yield 22.5 g (16.6%) of pure product.

### Step-3 Synthesis of Compound 4:

Formation of the Grignard reagent. To a stirred solution of magnesium (Mg) (25.0 g, 1.03 mol) in ether (600 ml) was added a small amount of iodine (I₂) under nitrogen (N₂) Atmosphere, and to that was added isopropyl bromide (124.0 g, 1.1 mol) in drop wise manner at ambient temperature. The reaction was stirred at ambient temperature for 1 hr. To a solution of compound 3 in ether under a nitrogen atmosphere was added the Grignard reagent in drop wise manner at 0° C. After completion of addition, the reaction was heated under reflux for 1 hr. The reaction was monitored by TLC. To the reaction mixture was then added dropwise saturated ammonium chloride (NH₄Cl) aq. at 0° C. The ether layer was separated from the reaction mixture and dried over anhydrous Na₂SO₄. The ether was then evaporated to yield a crude oil which was purified by silica gel column chromatography using Hexane & Ethyl acetate to yield 10.0 g (35.6%) of pure product.

### Step-4 Synthesis of Compound 5:

To the stirred solution compound 4 (10.0 g, 0.045 mol) in MDC (100 ml), was added p-toluenesulfonic acid (PTSA) (9.4 g, 0.05 mol) at ambient temperature, and stirred for an additional 24 hr at ambient temperature. The reaction was monitored by TLC. Water (200 ml) was then added and the organic layer separated and dried over anhydrous Na₂SO₄. MDC was filtered out and distilled out completely to get crude oil (9.5 g) which was purified by column chromatography using hexane and ethyl acetate to get 7.0 g (76.2%) of pure product.

Step-5 Synthesis of Compound 6: 2-(3-methyl)secbutyl-6-isopropylphenol:

A solution of compound 5 (7 g, 0.034 mol) in tetrahydrofuran (THF) (70 ml) in Parr apparatus bottle was charged. Ten percent palladium on carbon (Pd/C) (7 g) in one lot at ambient temperature was charged. Hydrogen (H₂) was applied at a pressure up to 4 to 5 kg in Parr bottle for 3 to 4 hrs. The reaction was monitored by TLC. The catalyst was then filtered off through a hyflow bed and the solvent was distilled out completely to afford a crude oil that was purified by repeated column chromatography by using hexane and ethyl acetate to afford the title compound. (2.90 g, 41.03%).

LCMS (Instrument: AB SCIEX-2000 API (Applied Biosystems). Mode: −Ve (Negative), Sample Preparation: 1.0 mg sample was dissolved in 50.0 ml methanol and spectra was recorded using sample infusion technique) also consistent with assigned structure. M−1=205.4; calculated mass=206.2.

NMR (Instrument: Bruker AVANCE 300 MHz. Solvent: deuterated chloroform (CDCl₃). Sample Preparation: 2.0 mg sample dissolved in 0.75 ml CDCl₃) consistent with assigned structure.

### Example 4

### Separation of Enantiomers by Chiral HPLC

Separation of the mixture of stereoisomers of 2-(3-methyl)secbutyl-6-isopropylphenol was achieved by chiral HPLC. 2-(3-Methyl)secbutyl-6-isopropylphenol (2 mg/ml in HPLC grade n-hexane) was injected onto a chiral HPLC column (Daicel, Inc. CHIRALCEL ODH 20×250 millimeter (mm), 5 micron (urn)). Separation was achieved by an isocratic gradient using HPLC grade n-hexane as the mobile phase at a flow rate of 12 ml/minute at ambient temperature. Peak detection was at 273 mm. 2-(3-Methyl)secbutyl-6-isopropylphenol showed two peaks in a 1:1 ratio corresponding to enantiomer 1 and enantiomer 2.

### Analytical Analysis of Isolated 2-(3-Methyl)Secbutyl-6-Isopropylphenol Enantiomers by Chiral Chromatography

2-(3-methyl)secbutyl-6-isopropylphenol, enantiomer 1 (0.2 mg/ml) is dissolved in HPLC grade n-hexane and injected onto a chiral HPLC column (Daicel, Inc. CHIRALCEL ODH 4.6×250 mm, 5 um), run with an isocratic gradient using HPLC grade n-hexane as the mobile phase at a flow rate of 1.0 ml/minute at ambient temperature. Peak detection was at 273 mm., and showed a retention time of 11.4 minutes, and a purity of >%95 of the isomer. Optical rotation: a²⁰_{D}=−3.11°. This enantiomer is identified herein as the (−) stereoisomer.

The analytical procedure is repeated for enantiomer 2. Peak detection was at 273 mm., and showed a retention time of 12.1 minutes, and a purity of >%95 of the isomer. This enantiomer is identified herein as the (+) stereoisomer.

### Example 5

### Synthesis of 2-secpentyl-6-isopropylphenol

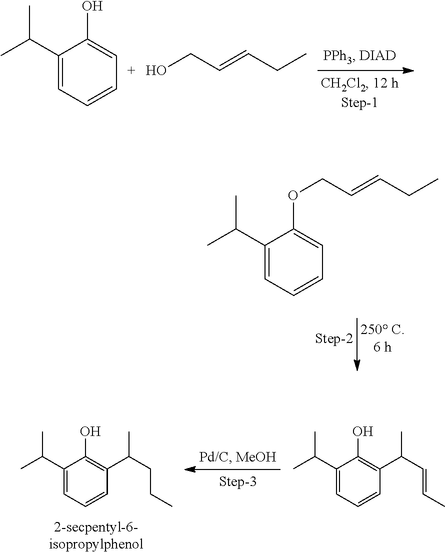

### Step-1:

To a solution of dry dichloromethane (CH₂Cl₂) (1 liter (L)) was added 2-isopropyl phenol (100 grams (g), 0.73 moles (mol)). The mixture was cooled to between 0° C. to −10° C. 2-Penten-1-ol (90 ml, 0.88 mol) was added to the reaction mixture, followed by triphenylphosphine (232 g, 0.88 mol) portionwise for about 1 hour (h). To this was added diisopropyl azodicarboxylate (DIAD) (172 milliliters (ml), 0.87 mol) dropwise over a period of 1 h. The reaction was brought to ambient temperature and stirred overnight. After the disappearance of the starting material as judged by thin layer chromatography (TLC), the reaction was diluted with dichloromethane (2 L), and washed with water (400 ml 2 times (×2)) and brine (200 ml). The organic layer was dried over anhydrous sodium sulfate (Na₂SO₄), filtered and concentrated to dryness to afford 95 g of crude product. The crude material (95 g) was added with petroleum ether (1 L) and stirred at ambient temperature for 1 h. The precipitated white solid (triphenylphosphine oxide) was filtered off and washed with pet ether (500 ml×2). The combined filtrate (˜2 L) was concentrated to obtain a yellow viscous liquid (˜85 g). This crude material was purified by silica gel column chromatography and the product was eluted at about 5% ethyl acetate in pet ether. The solvent was evaporated to afford 70 g of the pure material. Yield=47%.

### Step-2:

The product of step-1 (70 g, 0.34 mol) was heated to 290° C. under nitrogen atmosphere for about 72 h. The reaction mixture was then extracted with ethyl acetate (EtOAc) (1 L) and washed with water (200 ml×2) and brine (200 ml). It was then dried over Na₂SO₄, filtered and evaporated to afford 50 g of product.

### Step-3:

To a solution of the product of step-2 (50 g, 0.24 mol) in dry methanol (MeOH) (500 ml) kept at 0° C. was added palladium on carbon (Pd\C) (5 g, 10 mol %) and hydrogenated in an autoclave under 5 kg pressure overnight. The reaction mixture was then filtered through CELITE (Celite Corporation) and evaporated to result in 45 g of the crude product. The crude material (˜45 g) was purified by silica gel column chromatography using 2% ethyl acetate in pet ether to give rise to 40 g of the title compound. Distillation afforded 2-secpentyl-6-isopropylphenol as a colorless liquid (28 g). Yield=40%, combined from steps 2 and 3.

¹HNMR of the product was consistent with the assigned structure.

The (−)-stereoisomer and the (+) stereoisomer can be isolated from racemic 2-secpentyl-6-isopropylphenol using methods known to those of skill in the art such as, for example, fractional crystallization, separation on a chiral column, or by formation of derivatives. An example of a chiral HPLC column is Daicel, Inc. CHIRALCEL ODH 20×250 millimeter (mm), 5 micron (μm). Suitable solvents for the separation include HPLC grade n-hexane as the mobile phase.

### Example 6

### Formulation

The following illustrates a representative dosage form containing a compound of the invention for therapeutic use.

| Ingredient | Batch Weight | | w/w % |
|---|---|---|---|
| Soybean Oil | 70 | g | 11.71 |
| Soybean Phospholipids | 8.4 | g | 1.41 |
| (Lipid S-75) | | | |
| Compound of the invention | 3.5 | g | 0.59 |
| Glycerine | 15.75 | g | 2.64 |
| Disodium Edetate | 0.035 | g | 0.01 |
| Sodium Hydroxide | | | |
| (pH adjustment) | | | |
| Subtotal | 97.685 | | |
| Sterile Water For Injection | 500 | mL | 83.66 |
| Total | 597.685 | | 100 |

### Example 7

### Formulation

The following illustrates a representative dosage form containing a compound of the invention for therapeutic use.

| Ingredient | Batch Weight | | w/w % |
|---|---|---|---|
| Soybean Oil | 70 | g | 11.66 |
| Soybean Phospholipids | 8.4 | g | 1.40 |
| (Lipid S-75) | | | |
| Compound of the invention | 6.0 | g | 1.00 |
| Glycerine | 15.75 | g | 2.62 |
| Disodium Edetate | 0.035 | g | 0.01 |
| Sodium Hydroxide | | | |
| (pH adjustment) | | | |
| Subtotal | 100.185 | | |
| Sterile Water For Injection | 500 | mL | 83.31 |
| Total | 600.185 | | 100 |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula (I): 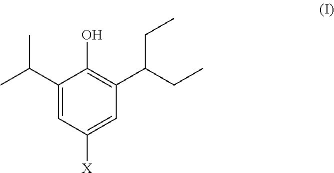
wherein X is H or F; or a pharmaceutically acceptable salt thereof.

2. A compound as claimed in claim 1, wherein X is H.

3. A pharmaceutical composition comprising a compound as claimed in claim 1, and a pharmaceutically acceptable carrier.

4. The pharmaceutical composition of claim 3 which is formulated for intravenous administration.

5. The pharmaceutical composition of claim 4 which is formulated as a lipid emulsion.

6. A method for inducing or maintaining general anesthesia in an animal comprising administering to the animal an effective amount of a compound as claimed in claim 1.

7. A method for promoting sedation in an animal comprising administering to the animal an effective amount of a compound as claimed in claim 1.

8-10. (canceled)

11. A process for the preparation of a compound as defined in claim 1, which comprises reducing a compound of general formula (II) 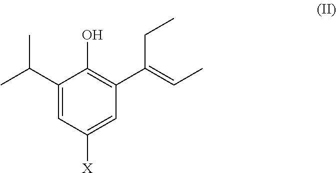
in which X is H or F, or a salt thereof, followed optionally by forming a pharmaceutically acceptable salt.

12. A pharmaceutical composition comprising a compound as claimed in claim 2, and a pharmaceutically acceptable carrier.

13. The pharmaceutical composition of claim 12 which is formulated for intravenous administration.

14. The pharmaceutical composition of claim 13 which is formulated as a lipid emulsion.

15. A process for the preparation of a compound as defined in claim 2, which comprises reducing a compound of general formula (II) 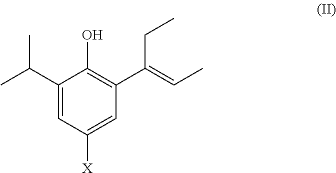
or a salt thereof, followed optionally by forming a pharmaceutically acceptable salt.

16. The method of claim 6, wherein X is H.

17. The method of claim 7, wherein X is H.

18. The process of claim 11, wherein the process comprises forming a pharmaceutical acceptable salt.

19. The process of claim 15, wherein the process comprises forming a pharmaceutical acceptable salt.
